(19) United States
(12) Patent Application Publication
(10) Pub. No.: US 2025/0082561 A1
(43) Pub. Date: Mar. 13, 2025

(54) IMPROVEMENTS IN OR RELATING TO ORGANIC COMPOUNDS

(71) Applicant: GIVAUDAN SA, VERNIER (CH)

(72) Inventors: Nathalie JOSET, Kemptthal (CH); Nathalie ALBRECHT, Kemptthal (CH); Gerhard BRUNNER, Kemptthal (CH); Martin Alan LOVCHIK, Kemptthal (CH); Marie MEUNIER, Pomacle (FR); Romain REYNAUD, Toulouse (FR); Amandine SCANDOLERA, Pomacle (FR)

(21) Appl. No.: 18/566,255

(22) PCT Filed: Jul. 15, 2022
(86) PCT No.: PCT/EP2022/069882

§ 371 Date: Dec. 1, 2023

(30) Foreign Application Priority Data
Jul. 15, 2021 (GB) 2110211.6

(51) Int. Cl. A61K 8/73; A61Q 5/00; A61Q 17/04; C08B 37/08

(52) U.S. Cl. CPC A61K 8/735; A61Q 5/002; A61Q 17/04; C08B 37/0072

(57) ABSTRACT

The present invention provides a hydroxypropyltrialkylammonium hyaluronate and/or salt thereof, having a degree of cationization of more than 1.4.

The present invention relates to hydroxypropyltrialkylammonium hyaluronate and/or salt thereof, having a degree of cationization of more than 1.4.

The desire to appear attractive is naturally rooted in modern consumers. Even as the ideal of attractiveness undergoes change over the course of time, it is universally accepted that the condition and appearance of our hair and skin is a significant contributor to an attractive outward appearance.

The structural framework of the skin is referred to as its extracellular matrix. It comprises a network of inter-meshed polymers, such as collagen and elastin, inside which skin cells are contained. It is responsible for the skin's mechanical properties, including firmness, strength, suppleness and elasticity. The physical signs of skin aging are a reflection of the condition of the skin matrix. More particularly, the weaker and less regular the matrix, the more wrinkles, roughness and sag the skin tends to have.

Skin hydration, essential to ensure the skin's suppleness, softness, tone and appearance, is a complex phenomenon. Water is a major constituent of our body and represents 60% of the body weight of adults. In the skin, water is mainly distributed in the dermis where it forms a semi-fluid gel with different structural proteins of the extracellular matrix. The epidermis and the stratum corneum contain very little water.

The epidermis is the external structure of the skin, the role of which is to ensure protection and exchanges with the environment. The stratum corneum is the outermost layer of the epidermis. It consists of the stacking of several layers (15 to 20) of corneocytes and is the end product of the epidermal keratinization process. Schematically, the stratum corneum comprises the corneocytes, rich in proteins and hydrophilic, and the intercorneocyte space, rich in lipids and hydrophobic. Corneocytes are anucleated cells that have lost their intracytoplasmic organelles. inside the corneocyte, a dense network of keratin filaments is dispersed within a matrix composed of another protein: filaggrin. The whole is surrounded by a very resistant envelope made of a protein wall lined with a lipid envelope.

Two layers of corneocytes are distinguished: the stratum corneum compactum, deep, where the corneocytes, connected to each other by the corneodesmosomes, provide a barrier function and a desquamating layer called stratum corneum disjunctum. Corneocytes can be assimilated to bricks forming a wall and linked together by a small number of desmosomes, appendages rich in proteins of the cell membrane. Between the corneocytes, the epidermal lipids creep in, synthesized by the keratinocytes of the thorny and granular layers, creating a “brick and cement” model. Epidermal lipids represent 10 to 30% of the volume of the stratum corneum. They are formed in the Golgi apparatus of keratinocytes and are then excreted by exocytosis into the extracellular space. Ceramides represent the majority of lipids in the stratum corneum (40%). This is a group of sphingolipids, essentially comprising sphingosines and various fatty acids such as linoleic acid. Ceramides bind the aqueous components of this complex lipid mixture. The main function of the intercellular lipids of the stratum corneum is to confer on this barrier layer the property of relative impermeability to water.

The level of skin hydration is expressed on the surface but the water comes mainly from the dermis which contains a large reserve of water of plasma origin. The dermis is made up of connective tissue characterized by an abundant extracellular matrix (ECM) located between the specific cells, fibroblasts and fibrocytes, which synthesized it. ECM consists of elastic fibers and collagen and an amorphous ground substance. This ground substance essentially contains hyaluronic acid (unsulfated glycosaminoglycans) and sulfated glycosaminoglycans which combine with a protein axis to form proteoglycans. The whole forms a compressible gel, which retains water like a sponge, also allowing the circulation of water and dissolved molecules. 20% to 40% of the total body water is contained in the extracellular matrix. The key molecule of skin hydration is therefore hyaluronic acid, capable of attracting and fixing up to 1000 times its weight in water. Understanding the metabolism of hyaluronic acid, its role within the skin and its interactions with other skin components is a more rational approach to consider the modulation of skin hydration.

Hyaluronic acid is a high molecular weight anionic polysaccharide which has the capacity to adopt various shapes and configurations depending on the pH, the salt content of the medium and the associated cations. Its role is essential in cell motility, adhesion, proliferation and tissue organization. It is the main component of the extracellular matrix, a key element in skin hydration. The functions of hyaluronic acid are multiple within the body. Within the extracellular matrix, much more than a passive structural component, hyaluronic acid is an intracellular regulator that plays a role in cell metabolism. There are, for example, receptors for hyaluronic acid on the surface of cells: CD44 and the receptor for mobility mediated by hyaluronic acid (RHAMM) are the two most important. They can induce cell mobilization after a cascade of intracellular signals and are themselves the substrates of many phosphokinases. Very recently, histological techniques have made it possible to demonstrate the presence of hyaluronic acid in the epidermis, whereas its presence was thought to be limited only to the dermis. The granular and prickly layers of the epidermis are the richest in hyaluronic acid, in the extracellular compartment. The basal layer also contains hyaluronic acid but in lesser quantity and in an intracellular situation. The hyaluronic acid contained in the basal layer of the epidermis is involved in the regulation of the cell cycle. The hyaluronic acid contained in the granular and prickly layers intervenes in the skin hydration by retaining the water molecules, coming from the dermis, contained in the extracellular matrix. The water is fixed by hyaluronic acid and retained by the hydrolipidic film.

Before arriving in the superficial layers of the epidermis, water is bound to the macromolecules of the ground substance of the dermis, mucopolysaccharides, hyaluronic acid and proteoglycans. Only a small part of this dermal water is free. Water diffuses from the dermis to the deep layers of the epidermis through the dermo-epidermal junction. The migration of this water from the deep layers to the superficial layers then calls on real water transport systems: aquaporins located in cell membranes. In the skin, it is almost exclusively type 3 aquaporins.

The water then gains the intercorneocyte spaces, retained by hyaluronic acid, and also penetrates inside the corneocytes to plasticize the keratin. Water is attracted and retained in the corneocytes thanks to a double phenomenon of osmosis and attraction by intracellular hygroscopic elements grouped together under the name of NMF (Natural Moisturizing Factors). These Natural Moisturizing Factors are agents naturally synthesized by the skin to capture water in the stratum corneum. They result from the degradation of filaggrin into amino acids, under the action of intracellular proteases, which, together with the detached and compacted keratin filaments, constitute a highly hydrophilic intracellular matrix. Free amino acids, pyrrolidone carboxylic acid, urea, lactates, sugars, trace elements and chloride are part of NMF. Pyrrolidone acid, urea and lactates have a hygroscopic action such that they can retain up to 70% of their weight in water, hence their strong moisturizing power. It is thanks to the hydrophobic nature of the lipids of the intercorneocyte spaces that water cannot leave the corneocyte. Finally, the surface hydrolipidic film, a natural emulsion formed of water and lipids, retains water on the surface of the skin, preventing insensible water loss (PIE). The aqueous part of the water-soluble fraction comes from skin perspiration and sweat secretion. The lipid part, liposoluble fraction, comes from sebum and the epidermal synthesis of lipids. The hydrolipidic film, essential for the suppleness of the skin, also helps prevent dehydration.

By its hygroscopic capacities and its high concentration in the dermis, hyaluronic acid plays a major role in skin hydration. It is present in the dermis, but also in the epidermis, where it retains water in the interkeratinocyte spaces. It is in fact found in the extracellular space of the upper layers of the epidermis; it fixes water molecules there, retained on the surface by the hydrolipidic film which limits its evaporation.

Mammalian and in particular human hair generally consists of three major components: The cuticle (the outer protective layer), the cortex (the massive core of the hair), and the medulla (a central soft protein core which is more common in thicker hair and particularly so in white hair). The main constituents of these structures are sulphur-rich proteins, lipids, water, melanin, and trace elements.

The cuticle is composed of keratins and usually consists of six to eight layers of flattened overlapping cells. Each cell contains several layers. The uppermost structure of each cuticle cell contains a thin proteinaceous membrane, the epicuticle or f-layer that is covered with a lipid layer. This layer lipid is covalently attached to the surface of the fiber. The epicuticle is hydrophobic. The cuticle's complex structure allows it to slide as the hair swells, and the f-layer imbues a considerable degree of water resistance. It is critical in protecting the hair and rendering it resistant to the influx and outflow of moisture. The normal cuticle has a smooth appearance, allowing light reflection and limiting friction between the hair shafts. It is primarily responsible for the luster and texture of the hair. The cuticle is a chemically resistant region surrounding the cortex in mammalian hair fibers. The cuticle may be damaged by environmental, mechanical, chemical, and heat sources. Chemical removal of the f-layer, particularly by oxidation during bleaching or perming, eliminates the first hydrophobic defense and leaves the hair more porous and vulnerable. If the cuticle is damaged there is little change in the tensile properties of hair; however, its protective function is diminished.

The cortex contributes almost all the mechanical properties of the hair, particularly strength and elasticity. The cortex consists of closely packed, spindle-shaped cells rich in keratin filaments comprising 400-500 amino acid residues paired together to form proto-filaments which make up a keratin chain. These are orientated parallel to the long axis of the hair shaft and embedded in an amorphous matrix of high sulphur proteins. The keratin chains have a large number of sulphur-containing cysteine bonds, which create a strong cross link between adjacent chains. These so-called disulfide bonds are critical in conferring shape, stability, and resilience to the hair shaft, and can only be broken by external oxidative chemical agents, such as those used for perming or relaxing. Weak hydrogen bonds link the keratin polypeptide chains together. These weaker bonds are easily overcome by water, rendering curly hair temporarily straight. The powerful disulfide bonds and weaker hydrogen bonds are crucial to hair health. The cortex also contains melanin granules, which are responsible for the color of the fiber.

The medulla is a soft proteinaceous core present in thicker and white hair. It has no known function in humans.

Today's consumers are offered a multitude of cosmetic products for the care of hair and skin. Generally, these products are in the form of leave-on or rinse-off formulations, depending on the intended application. Skin care products include, for instance, creams and lotions containing water for moisturizing the skin and fats and lipids for re-greasing it. Hair care products include, for instance, shampoos and conditioners for cleansing, moisturizing and UV-protecting the hair.

As we age, we lose collagen and hyaluronic acid naturally, so the skin becomes dehydrated more easily. Also, harsh weather, heaters during the wintertime, certain skin-care products, and underlying skin conditions can cause tiny breaks in the protective skin barrier, allowing water to escape. This is why a skin-care regimen with moisturizing products can be extra beneficial.

Hydrating skin-care ingredients including hyaluronic acid, glycerin, colloidal oatmeal, urea, propylene glycol, and sorbitol all act as humectants that attract water to the skin in an effort to hydrate it. These ingredients are widely used in products such as moisturizers, eye creams, and serums.

Hyaluronic acid is a sugar molecule that occurs naturally in the skin, and it helps to bind water to collagen, trapping it in the skin, so that skin can appear plumper, dewier, and more hydrated. It penetrates easily, which is why it works so well when applied topically. Additional perks of hyaluronic acid include its lightweight, watery nature and ability to lock in moisture from the environment and deeper dermis to fully hydrate the skin. Hyaluronic acid is not a moisturizer (it is a humectant), but it helps by pulling moisture in from the environment.

Hyaluronic acid is a compound naturally produced in the dermis of the skin, and is continuously degraded by an enzyme called hyaluronidase. Thus, with age, the ratio between hyaluronic acid synthesized by the cells and hyaluronic acid degraded by hyaluronidase decreases. This causes a reduction in dermal moisturization and progressive sag, which leads to the appearance of wrinkles. The skin-softening and moisturizing effects of hyaluronic acid are known in the art.

Hair is also often subjected to a wide variety of insults that can cause damage. These include shampooing, rinsing, drying, heating, combing, styling, perming, coloring, exposure to the elements, etc. Thus, hair is often in dry, rough, lusterless or frizzy due to abrasion of the hair surface and removal of the hair's natural oils and other natural conditioning and moisturizing.

Hyaluronic acid is also beneficial for hair: It hydrates the hair, reduces frizziness, plumps the hair, and hydrates the scalp. The humectant-binding properties of hyaluronic acid perform similarly on hair fibers they do on the skin, allowing the hair fibers to retain and seal moisture from products.

It also helps to seal the cuticle, which prevents unwanted moisture from entering it, leading to frizzy hair and shrinkage in curly hair textures.

There is, however, an important drawback:

The surface of hair and skin is normally negatively charged. Hyaluronic acid is normally also negatively charged due to the presence of anionic functional groups (in particular, carboxyl groups). Therefore, when treating hair or skin, hyaluronic acid and the hair/skin repel each other since their surfaces are negatively charged.

Therefore, hyaluronic acid normally adheres to the surface of hair or skin to only a small extent, rendering the treatment less effective.

In order to overcome this drawback, several groups have previously proposed to attach cationic groups to the hyaluronic acid backbone, thereby producing a cationized hyaluronic acid or salt thereof. Such cationized hyaluronic acid derivatives and salts thereof include, but are not limited to hydroxypropyltrimonium hyaluronate and salts thereof.

For example, US 2009/0281056 discloses a method for preparing a cationized hyaluronic acid—e.g. hydroxypropyltrimonium hyaluronate—wherein at least part of the hydroxylic hydrogen atoms of hyaluronic acid are replaced with a group having a quaternary ammonium cationic group. To this end, hyaluronic acid is reacted with a cationizing agent, such as glycidyltrialkylammonium halide.

For example, U.S. Pat. No. 8,410,076 relates to a cationized hyaluronic acid and/or a salt thereof—e.g. hydroxypropyltrimonium hyaluronate—which includes a quaternary ammonium group-containing group and has a degree of cationization of 0.15 to 0.6. This document further explains that, if the cationized hyaluronic acid and/or a salt thereof has a degree of cationization of less than 0.15, adhesion of the cationized hyaluronic acid and/or a salt thereof to hair or skin may decrease to a large extent, such that a sufficient moisturizing effect may not be obtained; and that, if the degree of cationization is more than 0.6, the cationized hyaluronic acid and/or a salt thereof adheres to hair or skin, but may not achieve a sufficient moisturizing effect and smoothness.

However, the above cationized hyaluronic acid derivatives and salts are not able to provide a long-lasting moisturizing effect even in rinse-off formulations.

It is therefore a problem of the present invention to provide a cosmetic moisturizing agent that is also effective in rinse-off application.

This problem is solved by the products, compositions and methods of the present invention as described below.

In a first aspect, the present invention relates to a hydroxypropyltrialkylammonium hyaluronate and/or salt thereof, having a degree of cationization of more than 1.4.

In a second aspect, the present invention relates to a method of preparing said hydroxypropyltrialkylammonium hyaluronate and/or salt thereof.

In a third aspect, the present invention relates to a cosmetic composition comprising said hydroxypropyltrialkylammonium hyaluronate and/or salt thereof.

In a fourth aspect, the present invention relates to the use of said hydroxypropyltrialkylammonium hyaluronate and/or salt thereof for hydration and/or UV protection and/or hair repair.

The present invention will be explained in more detail below.

The hydroxypropyltrialkylammonium hyaluronate and/or salt thereof of the present invention has a degree of cationization of more than 1.4.

It has been found that this cationized hyaluronic acid derivative is able to provide a long-lasting moisturizing effect, both to skin and to hair, and even in rinse-off applications. It is therefore a highly effective cosmetic active.

In particular, it was found to have a better adhesion to hair and to skin than commercially available hyaluronic acid derivatives with a lower degree of cationization, thereby providing a longer-lasting hydration effect.

It is the first time that a hyaluronic acid derivative has been found to be highly effective also in rinse-off applications, allowing for much broader formulation options than conventional materials.

Furthermore, the hydroxypropyltrialkylammonium hyaluronate and/or salt thereof of the present invention has been found to have a UV-protection effect, again not only in leave-on but also in rinse-off applications.

Furthermore, the hydroxypropyltrialkylammonium hyaluronate and/or salt thereof of the present invention has been found to have a hair repair effect after chemical treatment.

The term “hydroxypropyltrialkylammonium” relates to a group or substituent having the following structure:

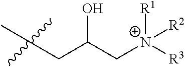

wherein R¹, R² and R³ are independently of one another linear or branched alkyl groups with 1 to 4 carbon atoms.

R¹, R² and R³ may be the same or different (e.g. R¹═R²═R³ or R¹═R²≠R³ or R¹≠R²≠R³), but preferably, they are all the same.

For example, R¹, R² and R³ are independently of one another selected from the group consisting of methyl, ethyl propyl, iso-propyl, and butyl.

The degree of cationization, as used throughout the application, corresponds to the average number of hydroxypropyltrialkylammonium groups attached to the hyaluronic acid per unit.

Hyaluronic acid is a polymer, wherein an N-acetyl-D-glucosamine and a D-glucoronic acid are bonded together to form one unit. Therefore, its general structure is the following (n representing the number of units):

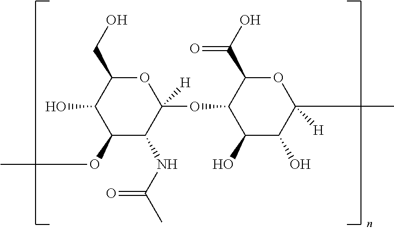

As can be seen from the above structure, each unit comprises several hydroxyl groups, including primary and secondary alcohols, as well as a carboxylic acid group. In addition, it also comprises an amide group. The hydroxypropyltrialkylammonium groups may be attached to any of these groups, i.e. replace the respective hydrogen atom.

The degree of cationization may be determined by means of NMR, IR and/or conductivity measurements. Further details are provided below in the examples section.

In an embodiment, the hydroxypropyltrialkylammonium hyaluronate and/or salt thereof is selected from the group consisting of hydroxypropyltrimonium hyaluronate and/or salt thereof; hydroxypropyltriethylammonium hyaluronate and/or salt thereof; hydroxypropyltripropylammonium hyaluronate and/or salt thereof; and hydroxypropyltributylammonium hyaluronate and/or salt thereof. Preferably, the hydroxypropyltrialkylammonium hyaluronate and/or salt thereof is hydroxypropyltrimonium hyaluronate and/or salt thereof.

The term “hydroxypropyltrimonium”, which is short for “hydroxypropyltrimethylammonium”, relates to a group or substituent having the following structure:

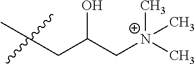 [SMILES: *CC(O)C[N+](C)(C)C]

In an embodiment, the hydroxypropyltrialkylammonium hyaluronate and/or salt thereof has a degree of cationization of at least 1.5, more preferably of at least 1.6, even more preferably of at least 1.7, and most preferably of at least 1.8. The degree of cationization may also be higher, for instance 1.9 or more, or 2.0 or more, or even 2.1 or 2.2 or more.

It has been found that a higher degree of cationization may lead to a better adhesion to hair and/or skin, thereby improving the deposition and the long-lastingness of the cosmetic active.

In theory, the degree of cationization may be up to 6.0. However, for cosmetic applications, it has been found that a degree of cationization of up to 3.0, more preferably of up to 2.8, and most preferably of up to 2.6 is most advantageous.

For instance, the hydroxypropyltrialkylammonium hyaluronate and/or salt thereof may have a degree of cationization of between 1.4 and 3.0, more preferably of between 1.6 and 2.4, and most preferably of between 1.8 and 2.0.

In an embodiment, the hydroxypropyltrialkylammonium hyaluronate and/or salt thereof is prepared from a hyaluronic acid or a salt thereof having an average molecular weight of about 10 kDa to about 200 kDa, more preferably of about 15 kDa to about 150 kDa, even more preferably of about 20 kDa to about 100 kDa, and most preferably of about 20 kDa to about 80 kDa.

Depending on the exact degree of cationization, the average molecular weight of the hydroxypropyltrialkylammonium hyaluronate and/or salt thereof may vary, so it seems more appropriate to define the weight of the hyaluronic acid or a salt thereof from which the hydroxypropyltrialkylammonium hyaluronate and/or salt thereof is derived. A person skilled in the art will easily be able to calculate the average molecular weight of the hydroxypropyltrialkylammonium hyaluronate and/or salt thereof based on the average molecular weight of the original hyaluronic acid or a salt thereof and the analytically determined degree of cationization as follows:

$${MW}_{catHA} = {MW}_{HA} + n * ( CatDeg * {MW}_{HPT} )$$

wherein
MW_{catHA} is the average molecular weight of the hydroxypropyltrialkylammonium hyaluronate and/or salt thereof;
MW_{HA} is the average molecular weight of the hyaluronic acid or salt thereof from which the hydroxypropyltrialkylammonium hyaluronate and/or salt thereof is derived;
n is the number of units of the hyaluronic acid or a salt thereof from which the hydroxypropyltrialkylammonium hyaluronate and/or salt thereof is derived;
CatDeg is the analytically determined degree of cationization;
MW_{HPT} is the molecular weight of the hydroxypropyltrialkylammonium group and/or salt thereof incorporated into the hydroxypropyltrialkylammonium hyaluronate and/or salt thereof; and
n may be calculated by dividing MW_{HA} by the molecular weight of one unit of the hyaluronic acid or a salt thereof.

For instance, the chloride salt of hydroxypropyltrimonium hyaluronate may be prepared from monosodium hyaluronate. In this case, the molecular weight of one unit is 401.3 g/mol, such that n=MW_{HA}/401.3 g/mol; and MW_{HPT} is 151.6 g/mol.

Thus, for example, the chloride salt of hydroxypropyltrimonium hyaluronate with a degree of cationization of 1.4 prepared from monosodium hyaluronate with an average molecular weight of 20 kDa will have an average molecular weight of about 30 kDa; and hydroxypropyltrimonium hyaluronate chloride with a degree of cationization of 2.5 prepared from monosodium hyaluronate with an average molecular weight of 50 kDa will have an average molecular weight of about 100 kDa.

Surprisingly, it has been found that a relatively low average molecular weight as defined above affords a particularly effective cosmetic active. For example, it exhibits a better adhesion to hair and penetrates deeper into the skin.

Furthermore, a relatively low average molecular weight facilitates the synthesis of the hydroxypropyltrialkylammonium hyaluronate and/or salt thereof. It has been found that hyaluronic acid with a lower average molecular weight reacts faster and requires less equivalents of the reagents in order to obtain the desired degree of cationization. Without being bound by theory, it is believed that the reactive sites of the hyaluronic acid with a higher average molecular weight are less accessible to the reagent molecules, both because of reduced flexibility of the longer polymer chains and of increased steric hindrance.

Throughout this application, unless otherwise indicated, equivalents of reagents are indicated relative to one repeating unit of the hyaluronic acid or the salt thereof that is used in the reaction.

In a particular embodiment, the hydroxypropyltrialkylammonium hyaluronate and/or salt thereof of the present invention is prepared from a hyaluronic acid or salt thereof with an average molecular weight of between 20 and 40 kDa, resulting—in the case of hydroxypropyltrimethyl-ammonium—in a hydroxypropyltrimonium hyaluronate and/or salt thereof with an average molecular weight of between 20 and 80 kDa.

Hydroxypropyltrialkylammonium hyaluronate as such has an overall positive charge, i.e. is cationic. Thus, typically, anionic counter ions will be present, for instance chloride or other halogen ions (e.g. bromide or iodide), hydroxide, phosphates, acetate, carboxylates (which optionally may be part of the hyaluronic acid backbone) or carbonates. Such counter ions may be introduced at any time during the synthesis of the hydroxypropyltrialkylammonium hyaluronate and/or salt thereof. Alternatively or in addition, counter ions may also be introduced from an ion exchange resin.

For instance, chloride counter ions may be introduced if a reagent bearing a chloride group has been used in the preparation of the hydroxypropyltrialkylammonium hyaluronate, or if a chloride salt (e.g. NaCl) has been used during the preparation, for instance during a washing or purification step.

In an embodiment, the hydroxypropyltrialkylammonium hyaluronate and/or salt thereof of the present invention comprises or consists of the chloride salt of hydroxypropyltrialkylammonium hyaluronate. Thus, some or all of the anionic counter ions may be chloride, and there may also be other anionic counter ions.

The hydroxypropyltrialkylammonium hyaluronate and/or salt thereof of the present invention may further also comprise additional cations, for instance alkali or alkaline earth metal cations (e.g. Na⁺, K⁺, Mg²⁺ or Ca²⁺).

In a further aspect, the present invention provides a method of preparing the hydroxypropyltrialkylammonium hyaluronate and/or salt thereof, in particular in the embodiments described above.

The method comprises the step of reacting hyaluronic acid and/or a salt thereof with a cationizing agent in the presence of a base. The cationizing agent is selected from the group consisting of 2,3-epoxypropyltrialkylammonium chloride, 2-chloro-3-hydroxypropyltrialkylammonium chloride, and mixtures thereof.

Alternatively, instead of or in addition to the chlorides, it is also possible to use 2,3-epoxypropyltrialkylammonium bromide, 2-chloro-3-hydroxypropyltrialkylammonium bromide, 2,3-epoxypropyltrialkylammonium iodide, 2-chloro-3-hydroxypropyltrialkylammonium iodide, or any mixtures of these reagents. Further alternatives include 2-chloro-3-hydroxypropyltrialkylammonium fluoride and 2-chloro-3-hydroxypropyltrialkylammonium acetate.

In an embodiment, the cationizing agent is selected from the group consisting of 2,3-epoxypropyltrimonium chloride, 2-chloro-3-hydroxypropyltrimonium chloride, and mixtures thereof. This leads to the formation of hydroxypropyltrimonium hyaluronate and/or salt thereof.

These cationizing agents are commercially available.

2,3-epoxypropyltrimonium chloride is sometimes also called glycidyltrimethylammonium chloride or (2,3-epoxypropyl) trimethylammonium chloride; thus, each of these terms refers to the same reagent.

2-chloro-3-hydroxypropyltrimonium chloride is sometimes also called 3-chloro-2-hydroxy-N,N,N-trimethylpropan-1-aminium chloride; each of these terms refers to the same reagent.

Alternatively, instead of or in addition to the chlorides, it is also possible to use 2,3-epoxypropyltrimonium bromide, 2-chloro-3-hydroxypropyltrimonium bromide, 2,3-epoxypropyltrimonium iodide, 2-chloro-3-hydroxypropyltrimonium iodide, or any mixtures of these reagents. Further alternatives include 2-chloro-3-hydroxypropyltrimonium fluoride and 2-chloro-3-hydroxypropyltrimonium acetate.

Preferably, 2,3-epoxypropyltrimonium chloride is used as the cationizing agent, as the reaction requires less base and has been found to run smoother.

Depending on the desired degree of cationization and the cationizing agent used, more or less equivalents of the cationizing agent should be used. For instance, about 5 to about 6 equivalents of 2,3-epoxypropyltrialkylammonium chloride may be used to obtain a degree of cationization of about 2.2; or about 5 to about 7 equivalents of 2-chloro-3-hydroxypropyltrialkylammonium chloride may be used to obtain a degree of cationization of about 1.6.

Suitable bases include, but are not limited to: inorganic bases, such as hydroxides, phosphates, hydrogenphosphates or carbonates of alkali and alkaline earth metals (e.g. NaOH, KOH, Ca(OH)₂, Mg(OH)₂, Na₃PO₄, K₃PO₄, Na₂HPO₄, K₂HPO₄, Na₂CO₃ or K₂CO₃); organic bases, such as, tributylamine, ethylene diamine, triethylamine, trimethylamine, tetra-n-butylammonium hydroxide or tetraethylammonium hydroxide.

Depending on the cationizing agent used, about 0.1 to about 15 equivalents of base should be used, more preferably about 1.0 to 5.0 equivalents, for example 1.3 equivalents. The use of larger amounts of base is disadvantageous, as the reaction has been found to be less efficient with higher volumes. If, on the other hand, a more concentrated base is used, then this may cause a partial hydrolysis of the hyaluronic acid polymer chain, leading to undesired side products and a lower yield of the desired product.

In particular, if 2-chloro-3-hydroxypropyltrimonium chloride or another 2-chloro-3-hydroxypropyltrialkylammonium is used, then an additional equivalent of base is necessary and the epoxide is prepared in situ, which allows for avoiding the handling of the toxic epoxide:

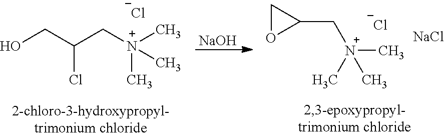

The cationization reaction is typically conducted at a basic pH, for instance at a pH of about 8 to about 14. In an embodiment, the cationization reaction is conducted at a pH of about 12 to about 13.

Suitable solvents include, but are not limited to, water, THF, DMSO, ethanol, methanol, isopropanol, acetone, acetonitrile, and combinations thereof. Preferably, and aqueous solvent is used, and in particular water.

The cationization reaction may be carried out at any suitable concentration that allows for obtaining a stirrable mixture, more preferably at a concentration where the hyaluronic acid and/or salt thereof is completely or at least essentially completely dissolved. For example, the reaction may be conducted at a concentration of about 0.01 to about 1.00 g/ml more preferably of about 0.05 to about 0.50 g/ml, of the hyaluronic acid and/or salt thereof.

A suitable reaction temperature may be about 10° C. to about 80° C., more preferably about 10° C. to about 40° C., for instance about 25° C. Higher temperatures may lead to hydrolysis of the hyaluronic acid polymer chain.

A suitable reaction time may be about 1 hour to about 6 days, for instance about 21 hours.

Optionally, the reaction may be stopped once a desired degree of cationization is reached or after a certain reaction time. For instance, the reaction may be stopped by the addition of a neutralizing agent, such as an acid, to bring the reaction to a neutral pH (e.g. a pH of less than 9, more preferably of less than 8, and most preferably of about 7). Suitable acids include, but are not limited to, inorganic acids, such as HCl or H₂SO₄, or organic acids, such as acetic acid, citric acid or oxalic acid.

By means of the synthetic procedure described above, a crude reaction mixture containing the desired hydroxypropyltrimonium hyaluronate and/or salt thereof is obtained, from which the hydroxypropyltrimonium hyaluronate and/or salt thereof is preferably isolated and purified.

The thus obtained product may be purified by any suitable method, for instance by means of dialysis, and in particular ultrafiltration, which was found to provide a very gentle and efficient purification with highly reproducible results. It also allows for removing any colored by-products.

Alternatively or in addition, an ion exchange resin (e.g. DOWEX MAC-3) may be used.

A further alternative is the precipitation of the product, e.g. by addition of ethanol or acetone to the reaction mixture.

The hydroxypropyltrialkylammonium hyaluronate and/or salt thereof may also be subjected to further treatment, e.g. to freeze drying or spray drying to obtain a powder.

The hydroxypropyltrialkylammonium hyaluronate and/or salt thereof may also be used in the form of an aqueous solution.

In a further aspect, the present invention relates to a cosmetic composition comprising the hydroxypropyltrialkylammonium hyaluronate and/or salt thereof as described above.

In particular, the present invention relates to a hair care or a skin care composition comprising the hydroxypropyltrialkylammonium hyaluronate and/or salt thereof as described above.

The hydroxypropyltrialkylammonium hyaluronate and/or salt thereof of the present invention is particularly suitable for cosmetic applications:
it provides a long-lasting moisturization
it strongly adheres to hair and skin, making it suitable for both leave-on and rinse-off formats
it provides efficient UV protection
it provides hair repair after chemical treatment

Hair care compositions have been used for many decades and for many different uses.

The hydroxypropyltrialkylammonium hyaluronate and/or salt thereof of the present invention may be used in all kinds of hair care compositions, for example in hair cleansing compositions, hair conditioning compositions, and hair styling compositions. Many of these compositions are typically water-based formulations.

Hair cleansing compositions are generally effective to remove soil from hair. The soil includes natural exudations from the scalp, environmental agents, and styling products. The soil can coat or deposit on the hair and scalp. Hair coated with such soil is typically greasy in feel and appearance, heavy to the touch, possibly malodorous, and generally unable to maintain a desired style. Known cleansing compositions typically include a combination of water and surface-active ingredients, such as soap or synthetic surfactants, and may also include a non-aqueous blend of starches. The combination of water and surface-active agents emulsifies the soil from the hair and scalp, allowing it to be rinsed away.

Cleansing compositions may also contain conditioning agents that deposit on the hair and scalp during rinsing with water. Such conditioning agents can include polymers, oils, waxes, protein hydrolysates, silicones, and mixtures and derivatives thereof. In addition, the conditioning composition can be a separate and different product from the cleansing composition.

Conditioning compositions that are known in the art are typically water-based formulations. However, there are also known conditioning compositions, which include at least one of silicones; animal, mineral or vegetable oils; waxes; petrolatums; and greases. The water-based conditioning compositions typically include substituted cationic waxes, fatty alcohols, cationic polymers, hydrolyzed proteins and derivatives thereof, and fragrances. Such conditioning formulations impart combability and manageability to the treated hair, thereby minimizing breakage during the styling process and resulting in shiny, healthy, and manageable hair. Conditioning compositions may also be effective to moisturize the hair. Subsequent drying and styling processes can include air drying or heating.

Nowadays, a multitude of different skin care products is available to the consumer.

The hydroxypropyltrialkylammonium hyaluronate and/or salt thereof of the present invention may be used in all kinds of skin care compositions, for example in hydrating and moisturizing compositions, anti-aging compositions, cleansing and refreshing compositions, or make-up compositions.

Skin care compositions of the present invention may contain one or more cosmetically acceptable excipients. Any excipients commonly used in the preparation of cosmetic preparations for use on the human skin may be employed in the present invention. Suitable excipients include, but are not limited to ingredients that can influence organoleptic properties, penetration of the skin, and the bioavailability of the cationized hyaluronic acid and/or salt thereof. More specifically, they include liquids, such as water, oils or surfactants, including those of petroleum, animal, plant or synthetic origin, such as and not restricted to, peanut oil, soybean oil, mineral oil, sesame oil, castor oil, polysorbates, sorbitan esters, ether sulfates, sulfates, betaines, glycosides, maltosides, fatty alcohols, nonoxynols, poloxamers, polyoxyethylenes, polyethylene glycols, dextrose, glycerol, digitonin, and the like.

The skin care composition may be in the form of a liposome composition, mixed liposomes, oleosomes, niosomes, ethosomes, milliparticles, microparticles, nanoparticles and solid-lipid nanoparticles, vesicles, micelles, mixed micelles of surfactants, surfactant-phospholipid mixed micelles, millispheres, microspheres and nanospheres, lipospheres, millicapsules, microcapsules and nanocapsules, as well as microemulsions and nanoemulsions, which can be added to achieve a greater penetration of the hydroxypropyltrialkylammonium hyaluronate and/or salt thereof.

The skin care composition may be produced in any solid, liquid, or semi-solid form useful for application to the skin topically or by transdermal application. Thus, these preparations of topical or transdermal application include, but are not restricted to, creams, multiple emulsions, such as and not restricted to, oil and/or silicone in water emulsions, water-in-oil and/or silicone emulsions, water/oil/water or water/silicone/water type emulsions, and oil/water/oil or silicone/water/silicone type emulsions, micro-emulsions, emulsions and/or solutions, liquid crystals, anhydrous compositions, aqueous dispersions, oils, milks, balsams, foams, aqueous or oily lotions, aqueous or oily gels, cream, hydro-alcoholic solutions, hydro-glycolic solutions, hydrogels, liniments, sera, soaps, face masks, serums, polysaccharide films, ointments, mousses, pomades, pastes, powders, bars, pencils and sprays or aerosols (sprays), including leave-on and rinse-off formulations.

For example, the hydroxypropyltrialkylammonium hyaluronate and/or salt thereof of the present invention may be used in anti-ageing products, moisturizing products, wash gels, lotions, cleansers, masks, hair or skin care products.

The cosmetic composition of the present invention may comprise the hydroxypropyltrialkylammonium hyaluronate and/or salt thereof in any suitable concentration sufficient to provide the desired effect. For example, it may comprise about 0.05% to about 1.0% of the hydroxypropyltrialkylammonium hyaluronate and/or salt thereof, for example about 0.1%. It has been found that a concentration of about 0.1% guarantees a high degree of adhesion on hair and skin and activation of biological efficacy. However, it would also be possible to include higher or lower concentrations.

The cosmetic composition of the present invention may further comprise additional cosmetic actives, such as anti-aging and anti-wrinkle actives, moisturizers, cleansers or hair conditioners.

For example, the hydroxypropyltrialkylammonium hyaluronate and/or salt thereof of the present invention may also be used in combination with hyaluronic acid and/or salt thereof and/or with other hyaluronic acid derivatives, such as e.g. acetates.

Alternatively or in addition, several hydroxypropyltrialkylammonium hyaluronates and/or salts thereof may be combined in one cosmetic composition. For instance, these may have different molecular weights and/or degrees of cationization and/or counter ions.

In a further aspect, the present invention also relates to the use of the hydroxypropyltrialkylammonium hyaluronate and/or salt thereof for hydration and/or UV protection and/or hair repair.

As has been described above and will be further displayed in the examples below, the hydroxypropyltrialkylammonium hyaluronate and/or salt thereof of the present invention provides a particularly effective hydration or moisturization, even in rinse-off applications, as well as UV protection and hair repair.

The present invention is further illustrated by means of the following non-limiting examples:

### EXAMPLE 1

### Preparation of Hydroxypropyltrimonium Hyaluronate Using (2,3-epoxypropyl)trimonium Chloride

A 350 ml, 4-neck sulfonation flask was equipped with a thermometer, cooler, over-head stirrer (Heidolph), bubble counter, and pH-meter and was flushed with nitrogen. It was then charged with 30 g of sodium hyaluronate (75 mmol; average MW of 41.5 kDa), 80 ml of deionized water and 72 ml of a 1.32 M aqueous sodium hydroxide solution (95 mmol). The mixture was stirred at 200 rpm for 1 h until a clear, light yellow solution was obtained.

72 g of (2,3-epoxypropyl)trimethylammonium chloride (449 mmol) was added to the mixture in one portion and the addition funnel was washed with 10 ml of deionized water. The internal temperature of the light yellow cloudy reaction mixture rose from 26° C. to 32° C. within one hour, before decreasing to 25° C. The light yellow clear solution was then stirred for 19 h at room temperature.

The viscous reaction mixture was transferred to a 2 l Schott bottle equipped with a magnetic stirrer and a pH-meter. The reaction flask was washed twice with 25 ml of deionized water. The mixture was then neutralized by the dropwise addition of 533 g of aqueous HCl (0.5 wt %) until a pH of 7.07 was reached. A clear yellow solution (825 ml) was obtained.

1175 ml of deionized water was added to dilute the mixture. The conductivity of the solution was 20.5 mS/cm at 22.9° C. The mixture was then ultrafiltrated for three days through two filtration VivaFlow200 units (MWCO: 10 kDa; polyether sulfone membrane; Sigma-Aldrich) until the filtrate conductivity reached 200 μS/cm. The mixture was then concentrated to reach 500 ml volume (turbid solution) before being freeze dried to obtain 35 g of hydroxypropyltrimonium hyaluronate chloride salt as defined above (48 mmol; 64% yield) in the form of white chips.

The cationization degree was determined by NMR (see example 3 below) as 2.18.

The molecular weight of the product was calculated based on the degree of cationization and assuming that the product still contains sodium and chloride ions (but the product may in fact also partially contain other counter ions, as described above):

$$[ 401.3 ] + 2.18 \times [ 151.6 ] = 731.86 g / mol$$

### EXAMPLE 2

### Preparation of Hydroxypropyltrimonium Hyaluronate Using 2-chloro-3-hydroxypropyltrimonium Chloride

Using the same equipment as described in Example 1, sodium hyaluronate (10 g, 24.92 mmol, 37.8 kDa) was mixed with 3-chloro-2-hydroxy-N,N,N-trimethylpropan-1-aminium chloride (40.6 ml, 150 mmol, 60% solution in water) at room temperature. Sodium hydroxide (16.77 ml, 181 mmol, 32% aqueous solution) was then added over 15 minutes. The exothermicity was controlled using a water bath. The mixture was stirred for 21 h at room temperature.

The yellow mixture was then poured into dialysis bags (30 cm/76 mm tubing, 14 kDa cut-off) closed with nodes and poured into 4.5 l water baths. The water baths were exchanged regularly over 48 h until a neutral pH was reached. The mixtures were filtered through neutralized DOWEX-MAC-3 ion exchange resin before being freeze-dried overnight to give 7.5 g of white hydroxypropyltrimonium hyaluronate chloride salt as defined above.

The degree of cationization was determined by ¹H-NMR (600 MHz) and found to be 1.47.

### EXAMPLE 3

### Determination of Cationization Degree

The degree of cationization was determined by a quantitative ¹H-NMR (600 MHz) by integration of the trimonium methyl signal versus the N-acetyl methyl signal.

In addition, a DOSY experiment was conducted to confirm that all trimonium groups were chemically bound to the hyaluronate, by comparing the diffusion constant to those stemming from the hyaluronate.

The degree of cationization may also be determined during the synthesis of the hydroxypropyltrimonium hyaluronate by means of an IR chemometric method. To this end, IR spectra of reference samples were associated with the degrees of cationization measured by NMR, using a partial least square method. An IR spectra of a new sample with an unknown degree of cationization can then be measured and, using the previously described model, the cationization degree can be assigned. This method was found to be independent from the presence of salt and more robust than the conductivity measurement.

Conductivity measurements provide a further alternative, but they are highly dependent on the work-up procedure, as the presence of any other ions can dramatically affect the results. For the conductivity measurements, several calibration curves were prepared for different dilutions of several samples with a known degree of cationization. This revealed a linear relationship between the conductivity and the concentration of the sample, with a higher degree of cationization leading to a steeper slope. In order to avoid interferences with other salts present in the solution (e.g. NaCl), the hydroxypropyltrimonium hyaluronate should be dialyzed prior to the measurement, and the use of an ion-exchange resin in the last purification step should be avoided.

### EXAMPLE 4

### Skin Adhesion Testing in Rinse-Off Application

### Skin Explants Preparation

Human fresh skin explant coming from two female donors (aged 35 and 57, respectively) having a breast reduction and lifting surgery, respectively, were used in this study. Skin explants were topically treated for 1 hour with one of the following six compositions:
Composition A: 1% of sodium hyaluronate with a molecular weight of 20-40 kDa (comparative example)
Composition B: 1% of hydroxypropyltrimonium hyaluronate prepared from the same sodium hyaluronate as used in Composition A, with a degree of cationization of 0.4 (comparative example)
Composition C: 1% of hydroxypropyltrimonium hyaluronate prepared from the same sodium hyaluronate as used in Composition A, with a degree of cationization of 1.4
Composition D: 1% of hydroxypropyltrimonium hyaluronate prepared from the same sodium hyaluronate as used in Composition A, with a degree of cationization of 2.4
Composition E: 1% of hydroxypropyltrimonium hyaluronate prepared from the same sodium hyaluronate as used in Composition A, with a degree of cationization of 2.0
Composition F: 1% of Hyaloveil®-P (ex Kewpie; hydroxypropyltrimonium hyaluronate with a molecular weight of 579 kDa and a degree of cationization of 0.6; comparative example)

Skin explants without any treatment were used as untreated condition. After 1 hour of treatment, skin explants were rinsed twice with sterile water, and excess water was softly absorbed with cleaning paper.

A part of skin explants were embedded in OCT for HABP staining on cryoslices, whereas the other part was freshly analyzed by Raman spectroscopy.

### HABP Staining

Deposition was evidenced with a fluorescent staining using Hyaluronic-acid Binding Protein (HABP) on 8 μm thickness cryoslices. Briefly, specific sites were saturated with successive bathes in avidin, biotin and 0.1% bovin serum albumin solutions. Biotinylated HABP was then incubated on cryoslices for 2 hours at room temperature, followed by rinses and another incubation with Streptavidin coupled to Alexa fluor 568 for 30 minutes at room temperature in the darkness. Samples were rinsed and assembled with coverslips and mounting medium. Images were collected with Axio Observed Inverted fluorescence microscope (Zeiss). Fluorescent intensity specific of hyaluronic acid deposition on skin stratum corneum was quantified.

### Raman Spectroscopy Analysis

The axial Z profiles were recorded directly on the skin samples. The Z profiles consist of an in-depth scanning through the skin. In this study, Raman spectra were collected at different focus points on skin surface, from Z=0 μm to Z=4 μm with a 2 μm step. A total of 35 Raman profiles were recorded (5 profiles per condition, n=5). The average spectra of the HA products was used as reference spectra.

### Results

In a first study, the skin adhesion properties of Compositions A, B and D were compared to an untreated control. The fitting coefficient of the hyaluronic acid (derivative) in the first layers of the stratum corneum were measured by Raman spectroscopy. The results are shown in the following table:

| | Mean (a.u) | Standard error of mean |
|---|---|---|
| Untreated | 0.00379 | 0.0019 |
| Composition A | 0.04428 | 0.0087 |
| Composition B | 0.09132 | 0.021 |
| Composition D | 0.1425 | 0.014 |

It was found that cationized hyaluronic acid samples (Compositions B and D) showed a significantly higher skin adhesion of +106% and +222%, respectively, compared to the non-cationized hyaluronic acid (Composition A).

Furthermore, Composition D of the present invention displayed a significantly higher skin adhesion than Composition B. Thus, a higher degree of cationization leads to a better skin adhesion.

In a second study, the skin adhesion properties of Compositions A, C, E, and F were compared to an untreated control. Adhesion of the hyaluronic acid (derivative) on the skin surface was revealed using HABP staining. The results are shown in the following table:

| | Mean (a.u) | Standard error of mean |
|---|---|---|
| Untreated | 6.821 | 0.515 |
| Composition A | 6.035 | 0.441 |
| Composition C | 8.85 | 0.751 |
| Composition E | 13.31 | 1.12 |
| Composition F | 5.343 | 0.280 |

It was found that the cationized hyaluronic acid samples of the present invention (Compositions C and E) showed a significantly higher skin adhesion of +47% and +121%, respectively, compared to the non-cationized hyaluronic acid (Composition A). Also, Composition E with the higher degree of cationization exhibited a significantly higher skin adhesion (+50%) than Composition C.

It was further found that the cationized hyaluronic acid samples of the present invention (Compositions C and E) showed a significantly higher skin adhesion of +67% and +149%, respectively, compared to the hyaluronic acid with a degree of cationization of 0.6 (Composition F). Composition F did not exhibit any skin adhesion property.

In conclusion, it was found that a higher degree of cationization led to a better skin adhesion.

### EXAMPLE 5

### Skin Hydration Testing in Rinse-Off Application: Comparison With HA of the Same Molecular Weight

### Skin Explants Preparation

Human fresh skin explants from a female donor (aged 22) having a breast reduction surgery were used in this study. Skin explants were topically treated for 5 minutes with one of the following two compositions:
Composition G: 0.1% of sodium hyaluronate with a molecular weight of 20-40 kDa (comparative example)
Composition H: 0.1% of hydroxypropyltrimonium hyaluronate prepared from the same sodium hyaluronate as used in Composition G, with a degree of cationization of 1.9

Skin explants without any treatment were used as untreated condition. After 5 minutes of treatment, skin explants were rinsed 5 times with sterile water.

These treatments were repeated once every day for 3 days. Skin hydration was analyzed on fresh skin explants by Raman spectroscopy at day 0 corresponding to skin explants without any treatment application, day 2 and day 3 corresponding to the second and third days following repeated treatments. Aquaporin and filaggrin immunostaining was performed on formalin-fixed and paraffin-embedded skin explants.

### Raman Spectroscopy Analysis

The axial Z profiles were recorded directly on the skin samples. The Z profiles consist of an in-depth scanning through the skin. Raman spectra were collected at different focus points on skin surface, from Z=0 μm to Z=30 μm with a 3 μm step. A total of 40 Raman profiles were recorded (4 profiles per condition n=4).

In a first step, the exact position of the stratum corneum surface was determined for each Raman profiles. In a second step, the lower limit of the SC was determined on the basis of the water content. This involved calculating the νOH/νCH ratio. This position corresponds to the maximum of νOH/νCH ratio. The average spectrum of the SC is calculated by taking into account all the spectra acquired on the SC. After data processing (base line correction, normalization, S/N ratio for spectral quality test), hydration parameters were calculated on the average spectra of SC of each profile.

For the assessment of skin hydration the integrated intensity of the OH vibration band on average SC spectra was calculated. This band represents the water content of the skin.

The spectral range used for the calculation was vOH: 3100-3600 cm⁻¹.

### Aquaporin and Filaggrin Immunostaining

Skin explants were cut into 4 μm thick slices, which were dewaxed, and antigenic retrieval was made overnight at 62° C. in EDTA buffer at pH 8.5 and in Citrate buffer at pH 6 for filaggrin and aquaporin-3, respectively. Non-specific sites were saturated with BSA at 2% in Tris buffer and primary antibodies were then incubated on skin slices overnight at 4° C. (anti-filaggrin antibody 1:100; anti-aquaporin-3 antibody 1:1000).

On the next day, the excess of antibody was washed three times with Tris buffer and a secondary antibody was incubated for 1 hour at room temperature: Hoechst 33342 1:5000 coupled to Alexa fluor 488 anti-mouse 1:100 for filaggrin or Alexa fluor 488 anti-rabbit 1:200 for aquaporin-3. Excess of antibody was washed three times with Tris buffer and mounting medium without DAPI was added with coverslips.

Pictures of the emitted fluorescent signal were taken with an inverted epifluorescent microscope (Axio Observer, Zeiss). Fluorescence intensity for each condition was measured using ImageJ software and results obtained with the treatments were compared to the untreated condition considered as the 100% control.

### Results

The skin hydration properties of Compositions G and H were compared to an untreated control after 2 and 3 days of application. The results after 2 and 3 days are shown in the following two tables, respectively:

| | Mean (% of | |
|---|---|---|
| | untreated D2) | Standard error of mean |
| Untreated D2 | 100 | 3.08 |
| Composition G D2 | 150 | 17.4 |
| Composition H D2 | 208 | 10.4 |

| | Mean (% of | |
|---|---|---|
| | untreated D3) | Standard error of mean |
| Untreated D3 | 100 | 14.8 |
| Composition G D3 | 217 | 11.5 |
| Composition H D3 | 262 | 25.0 |

After 2 and 3 days of application, a decrease of the basic skin hydration in the untreated condition relative to day 0 was observed, showing that culture condition induce a progressive loss of hydration in skin explants.

By applying Compositions G and H, respectively, the skin hydration was significantly improved relative to the untreated condition. Composition H of the present invention exhibited a significantly higher efficacy of +58% compared to Composition G at day 2; and an increase of +45% compared to Composition G at day 3.

In order to understand the difference of skin hydration between Compositions G and H on day 2, an immunostaining on aquaporin-3, a channel involved in water circulation into the skin and directly linked to skin moisturizing, was performed. It was found that Composition G had no significant effect on aquaporin-3 expression relative to the untreated condition, while Composition H significantly increased its expression by +16% relative to the untreated condition and also with a significant effect relative to Composition G:

| Aquaporin-3 expression | Mean (a.u.) | Standard error of mean |
|---|---|---|
| Untreated D2 | 38.1 | 2.46 |
| Composition G D2 | 40.9 | 2.00 |
| Composition H D2 | 44.2 | 1.43 |

Furthermore, the impact of each composition on the skin barrier function was analysed through the expression of filaggrin. It was found that Composition G had no effect on filaggrin expression, while Composition H significantly increased its expression by +35% versus untreated condition and by +36% versus Composition G.

| Filaggrin expression | Mean (a.u.) | Standard error of mean |
|---|---|---|
| Untreated D2 | 16.2 | 0.75 |
| Composition G D2 | 16.1 | 1.55 |
| Composition H D2 | 21.9 | 1.58 |

Thus, it was shown that Composition H is able to improve skin hydration in rinse-off application thanks to a biological effect regarding aquaporin-3 and filaggrin upregulation.

### EXAMPLE 6

### Skin Hydration Testing in Rinse-Off Application: Comparison With HA of High Molecular Weight

### Skin Explants Preparation

Human fresh skin explant coming from a woman donor (40 years old) having an abdominal surgery were used in this study. Skin explants were topically treated for 5 minutes with Composition H (see Example 5 above; containing 0.1% of hydroxypropyltrimonium hyaluronate) or Composition I (containing 0.1% of sodium hyaluronate with a molecular weight of 1000 to 1400 kDa) as a moisturizing control, and compared to an untreated condition. After 5 minutes of treatment, skin explants were rinsed 5 times with sterile water. These rinsing treatments were repeated every day for 2 days. Skin hydration was analyzed on fresh skin explants by Raman spectroscopy at day 0corresponding to skin explants without any treatment application, and on day 2 corresponding to the second day following repeated treatments.

### Raman Spectroscopy Analysis

The same analysis was done as in Example 5 above.

### Results

It was found that Composition I is not able to provide a moisturizing effect to the skin in rinse-off condition, while Composition H according to the invention increased skin hydration by +66% compared to the untreated condition and with a significant effect compared to Composition I.

| Skin hydration | Mean (a.u.) | Standard error of mean |
|---|---|---|
| Untreated D2 | 34.9 | 1.50 |
| Composition I D2 | 44.9 | 2.93 |
| Composition H D2 | 57.7 | 2.95 |

### EXAMPLE 7

### Skin Hydration Testing in Leave-On Application: Comparison With HA of the Same Molecular Weight

### Skin Explants Preparation

Human fresh skin explants coming from a female donor (aged 35) having a breast reduction surgery were used in this study. Skin explants were topically treated for 8 and 24 hours with one of the following four compositions:
Composition G: 0.1% of sodium hyaluronate with a molecular weight of 20-40 kDa (comparative example)
Composition J: 0.1% of hydroxypropyltrimonium hyaluronate prepared from the same sodium hyaluronate as used in Composition G, with a degree of cationization of 1.4
Composition K: 0.1% of hydroxypropyltrimonium hyaluronate prepared from the same sodium hyaluronate as used in Composition G, with a degree of cationization of 2.0
Composition L: 0.1% of Hyaloveil®-P (ex Kewpie; hydroxypropyltrimonium hyaluronate with a molecular weight of 579 kDa and a degree of cationization of 0.6; comparative example)

Skin explants without any treatment were used as untreated condition. After each incubation time, the excess of product was softly absorbed with cleaning paper and skin explants were freshly analyzed by Raman spectroscopy.

### Raman Spectroscopy Analysis

The axial Z profiles were recorded directly on the skin samples. The Z profiles consist of an in-depth scanning through the skin. Raman spectra were collected at different focus points on skin surface, from Z=0 μm to Z=30 μm with a 3 μm step. A total of 40 Raman profiles were recorded (4 profiles per condition n=4).

In a first step, the exact position of the SC surface was determined for each Raman profile. In a second step, the lower limit of the SC was determined on the basis of the water content. This involved calculating the νOH/νCH ratio. This position corresponds to the maximum of μOH/μCH ratio. The average spectrum of the SC was calculated by taking into account all the spectra acquired on the SC. After data processing (base line correction, normalization, S/N ratio for spectral quality test), hydration parameters were calculated on the average spectra of SC of each profile.

For the assessment of skin hydration, the integrated intensity of the OH vibration band on average SC spectra was calculated. This band represents the water content of the skin.

The spectral range used for the calculation is νOH: 3100-3600 cm⁻¹.

### Results

The skin hydration properties of Compositions G, J, K and L were compared to an untreated control after 8 and 24 hours of application. The results are shown in the following table:

| | 8 hours | | 24 hours | |
|---|---|---|---|---|
| | Mean | Standard error | Mean | Standard error |
| | (a.u) | of mean | (a.u) | of mean |
| Untreated | 37.39 | 1.38 | 26.62 | 1.71 |
| Composition G | 51.93 | 1.09 | 45.98 | 2.24 |
| Composition J | 56.44 | 2.29 | 50.02 | 2.07 |
| Composition K | 91.99 | 5.72 | 69.82 | 4.35 |
| Composition L | 54.05 | 3.98 | 62.25 | 0.88 |

After 8 hours, Composition K displayed a significant improvement of skin hydration compared to Compositions G, J and L.

After 24 hours, Compositions G, J and L progressively improved skin hydration relative to untreated condition. But the skin hydration with Composition K was still significantly better than with the others.

Thus, it was demonstrated that a higher degree of cationization accelerates skin hydration and delivered a prolonged hydration.

### EXAMPLE 8

### Skin Hydration Testing in Leave-On Application: Comparison With HA of High Molecular Weight

### Skin Explants Preparation

Human fresh skin explant coming from a woman donor (40 years old) having an abdominal surgery were used in this study. Skin explants were topically treated for 8, 24 and 72 hours with either Composition H (of the invention) or Composition I (control) as described in Example 6 above.

Skin explants without any treatment were used as untreated condition. After each incubation time, the excess of product was softly absorbed with cleaning paper and skin explants were freshly analysed by Raman spectroscopy.

### Raman Spectroscopy Analysis

The same analysis was done as in Example 7 above.

### Results

Regarding skin moisturizing in leave-on application compared to a high molecular weight moisturizing hyaluronic acid (Composition I), a significantly better skin hydration was detected when using cationized hyaluronic acid (Composition H) after 8 hours of treatment: Composition H exhibited a +95% higher efficacy than Composition I. After 24 and 72 hours, the two compositions were found to have almost the same efficacy. Therefore, the composition of the invention is able to provide a hydration effect much faster.

| | 8 hours | | 24 hours | | 72 hours | |
|---|---|---|---|---|---|---|
| | Mean | Standard error | Mean | Standard error | Mean | Standard error |
| | (a.u.) | of mean | (a.u.) | of mean | (a.u.) | of mean |
| Untreated | 40.3 | 13.27 | 59.2 | 10.37 | 56.1 | 3.38 |
| Composition I | 90.3 | 5.51 | 124.0 | 10.67 | 182.3 | 17.12 |
| Composition H | 128.5 | 8.87 | 136.9 | 11.53 | 212.0 | 46.42 |

### EXAMPLE 9

### Hair Adhesion Testing in Rinse-Off Application

Human hair locks were soaked in water bath and then massaged for 2 minutes with one of the following three shampoo compositions:
Composition M: 0.1% of sodium hyaluronate with a molecular weight of 20-40 kDa (comparative example)
Composition N: 0.1% of hydroxypropyltrimonium hyaluronate prepared from the same sodium hyaluronate as used in Composition L, with a degree of cationization of 2.0
Composition O: Placebo with no hyaluronic acid or hyaluronic acid derivative (comparative example)

The full formulations were as follows:

| | Compo- | Compo- | Compo- |
|---|---|---|---|
| | sition | sition | sition |
| | M (% | N (% | O (% |
| | w/v) | w/v) | w/v) |
| SLES TEXAPON NSO 27% | 28.93 | 28.93 | 28.93 |
| DEHYTON KCOS | 12 | 12 | 12 |
| PHENOXYETHANOL | 0.6 | 0.6 | 0.6 |
| Perfume ALOE AQUA EAE08468/00 | 0.2 | 0.2 | 0.2 |
| WATER | 57.67 | 57.67 | 57.77 |
| Sodium hyaluronate with a molecular | 0.1 | / | / |
| weight of 20-40 kDa | | | |
| Hydroxypropyltrimonium hyaluronate | / | 0.1 | / |
| with a degree of cationization of 2.0 | | | |

A control hair lock was kept untreated with any shampoo. After shampoo massage, hair locks were rinsed 3 times in a controlled volume of water, and dried with a hair-dryer for 3 minutes.

### Results

Hair adhesion of Compositions M and N was compared to that of placebo Composition O. Alcian Blue staining allowed for visualization of the hyaluronic acid (derivatives) on the hair fibers. The results are shown in the following table:

| | Mean | Standard Deviation |
|---|---|---|
| Untreated | 6.308 | 5.329 |
| Composition M: | 6.75 | 4.432 |
| Composition N: | 13.08 | 5.139 |
| Composition O: | 8.615 | 4.194 |

It was found that the non-cationized hyaluronic acid of Composition M did not bind to hair fibers when compared to the placebo, while the cationized hyaluronic acid of Composition N showed a significantly higher hair adhesion of +52% compared to the placebo Composition O and of +107% compared to the Untreated, respectively.

### EXAMPLE 10

### Hair Repair Testing in Rinse-Off Application Using Scanning Electronic Microscopy (SEM) and Atomic Force Microscopy (AFM)

### Surface Visualization by Scanning Electronic Microscopy (SEM)

In order to evaluate if the adhesion to the hair fibers could form a protective layer against UV irradiation, human hair locks were soaked in water bath and then massaged for 2 minutes with one of the following two shampoo compositions, prior to UV treatment:
Composition P: Placebo with no hyaluronic acid or hyaluronic acid derivative (comparative example)
Composition Q: 0.1% of hydroxypropyltrimonium hyaluronate prepared from sodium hyaluronate with a molecular weight of 20-40 kDa, with a degree of cationization of 1.9

The full formulations were as follows:

| | Composition | Composition |
|---|---|---|
| | P (% w/v) | Q (% w/v) |
| SLES TEXAPON NSO 27% | 28.93 | 28.93 |
| DEHYTON KCOS | 12 | 12 |
| PHENOXYETHANOL | 0.6 | 0.6 |
| Perfume ALOE AQUA EAE08468/00 | 0.2 | 0.2 |
| WATER | 57.77 | 57.67 |
| hydroxypropyltrimonium hyaluronate | / | 0.1 |
| with a degree of cationization of 1.9 | | |

A control hair lock was kept untreated, without any shampoo. After shampoo massage, hair locks were rinsed 3 times in a controlled volume of water, and dried with a hair-dryer for 3 minutes.

One half of each hair lock was treated with a single UV-irradiation: 20 J/cm² UVA and 0.6 J/cm² UVB. The hair surface was then visualized using Scanning Electronic Microscopy.

### Malondialdehyde (MDA) and Total Protein Content Measurements

In order to quantify the observations made in SEM, MDA and total protein content were measured: MDA is a marker of lipids peroxidation; and UV-irradiation significantly increases MDA content in hair fibers, translating an oxidative stress following UV-irradiation. And the total protein content allows for assessing hair damage: When a biological sample is damaged, proteins are degraded to shorter proteins, thereby increasing the total protein content in the sample.

### Hair Roughness by Atomic Force Microscopy (AFM)

The measurements were carried out using the Nanowizard III atomic force microscope using an MLCT tip (Bruker). Three 25 μm×25 μm acquisitions were performed on three hair samples (about 50 μm in diameter) from each condition.

Nanoscale analysis was performed using JPK Data Processing software. The topography of the surfaces, the roughness and the mechanical properties of the hair (adhesion and elasticity) were measured.

Roughness measurements were made on the height map. A curve showing the average roughness as a function of the surface (5 μm×5 μm, 12.5 μm×12.5 μm and 25 μm×25 μm) allowed for the analysis of the roughness of each sample and comparing them.

### Results

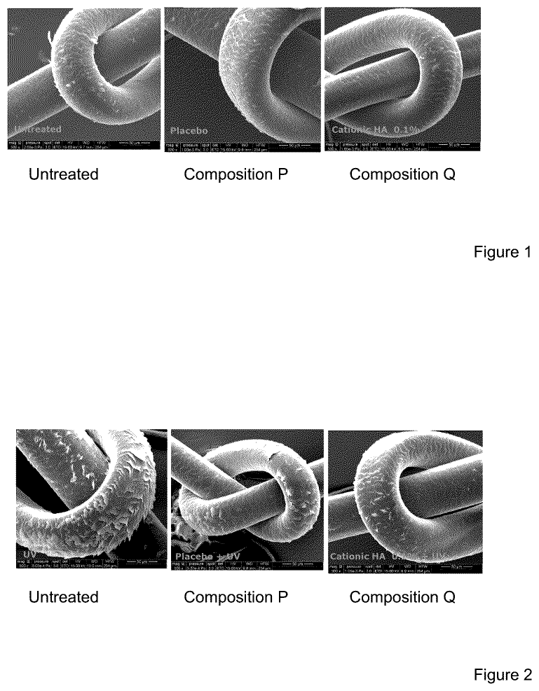

The hair surface was visualized before and after UV treatment: FIG. 1 shows the SEM images of hair fibers washed with either one of Compositions P and Q or untreated, before UV treatment; and FIG. 2 shows the SEM images of hair fibers washed with either one of Compositions P and Q or untreated, after UV treatment.

As can be seen from FIG. 1, in the basic condition, prior to UV irradiation, the hair fiber treated with Composition Q of the present invention seems to be slightly smoother than the other three. This is a first confirmation that the hydroxypropyltrimonium hyaluronate is deposited on the hair fiber and smoothens the surface.

After UV irradiation, a detachment of the hair's keratin scales was clearly observed in the untreated condition on the very left of FIG. 2, evidencing the negative impact of UV irradiation on the keratin structure. There was only a slight improvement with Composition P (placebo) compared to the untreated sample, while a clear smoothing effect was observed for Composition Q according to the invention.

These results suggest that hydroxypropyltrimonium hyaluronate of the present invention is able to protect the hair fibers against UV irradiation.

The results of the MDA measurements are shown in the following table:

| | Mean | Standard Deviation |
|---|---|---|
| Untreated, prior to UV irradiation: | 8.27 | 0.84 |
| Untreated, after UV irradiation: | 17.33 | 0.44 |
| Composition P, after UV irradiation: | 17.53 | 0.30 |
| Composition Q, after UV irradiation: | 14.37 | 0.81 |

As can be seen from the above, treatment with Composition Q significantly reduced the MDA content in hair fibers, showing a protective effect against UV-irradiation for this composition.

The results of the total protein content measurements are shown in the following table:

| | Mean | Standard Deviation |
|---|---|---|
| Untreated, prior to UV irradiation: | 1303 | 177 |
| Untreated, after UV irradiation: | 2325 | 225 |
| Composition P, after UV irradiation: | 1882 | 98 |
| Composition Q, after UV irradiation: | 1577 | 144 |

As can be seen from the above, UV irradiation let to a significant increase of the total protein content, whereas treatment with Composition P provided a protection for the hair fiber.

While SEM showed the hair structure in a global view, AFM allowed to focus on smaller scales, such as 5 μm×5 μm scale. It revealed an increase of keratin scales thickness, but also a kind of roughness on the scales' surface after UV irradiation in comparison with the untreated condition.

The application of a placebo shampoo (Composition P) did not improve the hair surface, and left a visible roughness on the scales' surface. But when the hair locks were treated with a shampoo containing cationized hyaluronic acid at 0.1% (Composition Q), a visible smoothing of the scales' surface was observed. This roughness was measured, and it was confirmed that UV irradiation significantly increases hair roughness by +31% compared to the untreated condition, with a similar effect for Composition P, while Composition Q significantly reduced the hair roughness by −29% versus the placebo condition.

| | | Standard error of |
|---|---|---|
| Hair roughness (nm) | Mean | mean |
| Untreated, prior to UV irradiation: | 68.7 | 4.41 |
| Untreated, after UV irradiation: | 90.0 | 5.17 |
| Composition P, after UV irradiation: | 87.3 | 3.68 |
| Composition Q, after UV irradiation: | 67.4 | 3.57 |

### EXAMPLE 11

### Hair Protection Testing in Rinse-Off Application Using Photonic Birefringence

### Analysis of Hair Protection Against UV Irradiation by Photonic Birefringence

Human hair locks were soaked in water bath and then massaged for 2 minutes with either one of the two shampoo compositions described in Example 10 above (Compositions P and Q, respectively).

A control hair lock was kept untreated with any shampoo. After shampoo massage, hair locks were rinsed 3 times in a controlled volume of water, and dried with a hair-dryer for 3 minutes. This successive washing-drying cycle was repeated 3 times.

Then, 1 cm of each hair lock was cut, spread in petri dishes and treated with 7 repetitions of UV-irradiation: 9 J/cm² UVA and 0.33 J/cm² UVB. This repetition of 3 shampoo and 7 UV-irradiation mimics hair care and daily global exposure to UV over one week.

Hair alteration was then analyzed using photonic birefringence: The more a hair shaft is altered (by UV, heat, chemical products . . . ), the more the photonic birefringence decreases compared to a non-treated hair shaft. On the other hand, if a hair shaft is protected against the treatment, then the photonic birefringence will increase.

### Results

The results are shown in the following table:

| Variation relative to native hair | Mean |
|---|---|
| Untreated, after UV irradiation: | −13.5% |
| Composition P, after UV irradiation: | −8.2% |
| Composition Q, after UV irradiation: | +5.8% |

As can be seen from the above, there was a significant decrease by −13.5% of photonic birefringence with UV irradiation relative to non-irradiated condition, confirming the deleterious effect of daily UV exposure on hair keratin structure. Treatment with the placebo Composition P did not provide a significant improvement, while Composition Q of the present invention was able to significantly improve photonic birefringence.

Therefore, the cosmetic composition of the present invention provides efficient UV protection to the hair.

### EXAMPLE 12

### Hair Repair Testing in Rinse-Off Application Using Photonic Birefringence

### Analysis of Hair Repair After Chemical Stress by Photonic Birefringence

Human hair locks were chemically treated by applying 3 times a bleaching solution containing 9% H₂O₂ and 3% Ammonium persulfate for 1 hour at 40° C. After this treatment, the hair locks were rinsed 3 times in water solution (200 ml) and then dried for 1 hour in an oven at 40° C. The hair locks were straightened using a hair straightener at 220° C. for 1 minute to increase the breakage of disulphide bonds and increase porosity (3 passages).

The hair locks were then soaked in a water bath and then massaged for 2 minutes with either one of the two shampoo compositions described in Example 10 above (Compositions P and Q, respectively).

A control hair lock was kept untreated. After shampoo massage, the hair locks were rinsed 3 times in a controlled volume of water, and dried with a hair-dryer for 3 minutes. This successive washing-drying cycle was repeated 3 times.

Hair alteration was then analyzed using photonic birefringence.

### Results

The results are shown in the following table:

| Variation relative to degraded hair | Mean |
|---|---|
| Composition P after chemical stress: | +5.6% |
| Composition Q after chemical stress: | +24.9% |

A slight repair effect was observed with composition P, with an increase of the photonic birefringence by +5.6% relative to degraded hair. However, this result is negligible in comparison with Composition Q of the present invention, which was able to significantly improve the photonic birefringence by +24.9%, with a significant effect also relative to Composition P.

Therefore, the cosmetic composition of the present invention provides efficient hair repair.

1. A hydroxypropyltrialkylammonium hyaluronate and/or salt thereof, having a degree of cationization of more than 1.4.

2. The hydroxypropyltrialkylammonium hyaluronate and/or salt thereof according to claim 1, wherein the hydroxypropyltrialkylammonium hyaluronate and/or salt thereof is selected from the group consisting hydroxypropyltrimonium hyaluronate and/or salt thereof; hydroxypropyltriethylammonium hyaluronate and/or salt thereof; hydroxypropyltripropylammonium hyaluronate and/or salt thereof; and hydroxypropyltributylammonium hyaluronate and/or salt thereof.

3. The hydroxypropyltrialkylammonium hyaluronate and/or salt thereof according to claim 2, wherein the hydroxypropyltrialkylammonium hyaluronate and/or salt thereof is hydroxypropyltrimonium hyaluronate and/or salt thereof.

4. The hydroxypropyltrialkylammonium hyaluronate and/or salt thereof according to claim 1 having a degree of cationization of at least 1.5.

5. The hydroxypropyltrialkylammonium hyaluronate and/or salt thereof according claim 1, wherein the hydroxypropyltrialkylammonium hyaluronate and/or salt thereof is prepared from a hyaluronic acid or a salt thereof having an average molecular weight of about 10 kDa to about 200 kDa.

6. The hydroxypropyltrialkylammonium hyaluronate and/or salt thereof according to claim 1, comprising or consisting of the chloride salt of hydroxypropyltrialkylammonium hyaluronate.

7. A method of preparing the hydroxypropyltrialkylammonium hyaluronate and/or salt thereof according to claim 1, the method comprising the step of: reacting hyaluronic acid and/or a salt thereof with a cationizing agent in the presence of a base, wherein the cationizing agent is selected from the group consisting of: 2,3-epoxypropyltrialkylammonium chloride, 2-chloro-3-hydroxypropyltrialkylammonium chloride, and mixtures thereof.

8. The method according to claim 7, wherein the cationizing agent is selected from the group consisting of: 2,3-epoxypropyltrimonium chloride, 2-chloro-3-hydroxypropyltrimonium chloride, and mixtures thereof.

9. The method according to claim 7, wherein about 1.5 to about 20 equivalents of the cationizing agent are used.

10. The method according to claim 7, wherein the reaction is conducted at a concentration of about 0.01 to about 1.00 g/ml, more preferably of about 0.05 to about 0.50 g/ml, of the hyaluronic acid and/or salt thereof.

11. A cosmetic composition comprising the hydroxypropyltrialkylammonium hyaluronate and/or salt thereof according to claim 1 and a suitable carrier.

12. The cosmetic composition according to claim 11, wherein the cosmetic composition is a hair care or a skin care composition.

13. A method of providing hydration and/or UV protection and/or hair repair, comprising the step of applying the hydroxypropyltrialkylammonium hyaluronate and/or salt thereof of claim 1 to epidermis or hair.

14. The hydroxypropyltrialkylammonium hyaluronate and/or salt thereof according to claim 4 having a degree of cationization of at least 1.5.

15. The hydroxypropyltrialkylammonium hyaluronate and/or salt thereof according to claim 4 having a degree of cationization of at least 1.6.

16. The hydroxypropyltrialkylammonium hyaluronate and/or salt thereof according to claim 4 having a degree of cationization of at least 1.7.

17. The hydroxypropyltrialkylammonium hyaluronate and/or salt thereof according claim 5, wherein the hydroxypropyltrialkylammonium hyaluronate and/or salt thereof is prepared from a hyaluronic acid or a salt thereof having an average molecular weight of about 15 kDa to about 150 kDa.

18. The hydroxypropyltrialkylammonium hyaluronate and/or salt thereof according claim 5, wherein the hydroxypropyltrialkylammonium hyaluronate and/or salt thereof is prepared from a hyaluronic acid or a salt thereof having an average molecular weight of about 20 kDa to about 100 kDa.

19. The method according to claim 7, wherein the reaction is conducted at a concentration of about 0.05 to about 0.50 g/ml, of the hyaluronic acid and/or salt thereof.
